(19) United States
(12) Patent Application Publication
(10) Pub. No.: US 2010/0311602 A1
(43) Pub. Date: Dec. 9, 2010

(54) Sequencing method

(75) Inventors: Samuel Levy, Rockville, MD (US); Susanne Goldberg, Germantown, MD (US); Karen Beeson, Rockville, MD (US)

Correspondence Address: VENABLE LLP, P.O. BOX 34385, WASHINGTON, DC, 20043-9998, US

(73) Assignee: J. Craig Venter Institute, Inc., Rockville, MD (US)

(21) Appl. No.: 12/311,780

(22) PCT Filed: Oct. 15, 2007
(86) PCT No.: PCT/US2007/021981

§ 371 Date: Apr. 13, 2009

Related U.S. Application Data
(60) Provisional application No. 60/851,292, filed on Oct. 13, 2006.

(51) Int. Cl. C40B 30/04; C12P 19/34; C07H 21/04

(52) U.S. Cl. 506/9; 435/91.5; 435/91.2; 536/23.1

(57) ABSTRACT

The present invention relates, e.g., to a method for isolating a DNA molecule of interest in a form suitable for sequencing at least a portion of the DNA by a high throughput sequencing method, comprising (a) digesting a double-stranded (ds) DNA molecule with two different restriction enzymes, A and B, to generate a ds form of the DNA molecule of interest, which is bounded by the two restriction enzyme cleavage products, and (b) attaching to each end of the DNA molecule of interest an adaptor molecule which comprises at one end a restriction enzyme cleavage site that is compatible with the restriction enzyme A or the restriction enzyme B cleavage product, and which also comprises a sequence and/or element that allows the DNA of interest to be sequenced with a high throughput sequencing apparatus. The method can be adapted for sequencing DNA with a variety of high throughput sequencing apparatuses, including machines manufactured by the 454, Illumina (Solexa Sequencing technology) and ABI (SOLiD™ Sequencing technology) companies. A method is also described for sequencing regulatory elements within a cell, comprising subjecting a collection of ds DNA molecules that are enriched for regulatory elements and that are generated by digestion with two restriction enzymes, A and B, which generate sticky ends, to an isolation method of the invention, and sequencing the collection of ds DNA molecules with a high throughput sequencing apparatus.

This application claims the benefit of the filing date of U.S. Provisional Application No. 60/851,292, tiled Oct. 13, 2006, which is incorporated by reference herein in its entirety.

Aspects of this invention were made with U.S. government support under Grant No. NHGRI Cooperative Agreement: 5 U54 HG003068-03 awarded by the National Human Genome Research Institute. The government has certain rights in the invention.

### FIELD OF THE INVENTION

This invention relates, e.g., to methods for isolating DNA molecules and for sequencing the isolated DNA molecules.

### BACKGROUND INFORMATION

The cis-acting sequence elements that participate in the regulation of a single metazoan gene can be distributed over 100 kilobase pairs or more. Combinatorial utilization of regulatory elements allows considerable flexibility in the timing, extent and location of gene expression. The separation of regulatory elements by large linear distances of DNA sequence facilitates separation of functions, allowing each element to act individually or in combination with other regulatory elements. Noncontiguous regulatory elements can act in concert by, for example, looping out of intervening chromatin, to bring them into proximity, or by recruitment of enzymatic complexes that translocate along chromatin from one element to another. Determining the sequence content of these cis-acting regulatory elements offers great insight into the nature and actions of the trans-acting factors which control gene expression, but is made difficult by the large distances by which they are separated from each other and from the genes which they regulate. The informational content of a gene does not depend solely on its coding sequence, but also on cis-acting regulatory elements, present both within and flanking the coding sequences. These include promoters, enhancers, silencers, locus control regions, boundary elements and matrix attachment regions, all of which contribute to the quantitative level of expression, as well as the tissue- and developmental-specificity of expression of a gene. Furthermore, the aforementioned regulatory elements can also influence selection of transcription start sites, splice sites and termination sites.

Identification of cis-acting regulatory elements has traditionally been carried out by identifying a gene of interest, then conducting an analysis of the gene and its flanking sequences. Typically, one obtains a clone of the gene and its flanking regions, and performs assays for production of a gene product (either the natural product or the product of a reporter gene whose expression is presumably under the control of the regulatory sequences of the gene of interest). A problem for this type of analysis is that the extent of sequences to be analyzed for regulatory content is not concretely defined, since sequences involved in the regulation of metazoan genes can occupy up to 100 kb of DNA. Furthermore, assays for gene products are often tedious and reporter gene assays are often unable to distinguish transcriptional from translation regulation and can therefore he misleading. Methods for identifying regulatory DNA sequences (particularly in a high-throughput fashion), collections of regulatory sequences, and databases of regulatory sequences would considerably advance the fields of genomics and bioinformatics.

### DESCRIPTION OF THE DRAWINGS

FIG. 1 illustrates schematically a method for isolating a collection of ssDNAs of interest, using defined adaptor molecules.

FIG. 2 shows agarose gel purification of digested DNA.

FIG. 3 shows the over-representation of NLA-hypersensitive sties in a region upstream of the CD34 gene.

FIG. 4 shows the mapping of three hypersensitive sites in an intron of the CD34 gene.

FIG. 5 shows the distribution of NLA-hypersensitive site and therefore putative regulatory fragments relative to all transcriptional start sites.

FIG. 6 shows a characterization of non-mapped fragments.

FIG. 7 diagrammatically illustrates an embodiment of the method. The “DNA of interest” is not drawn to scale; it is generally considerably longer than the length of the adaptor molecules.

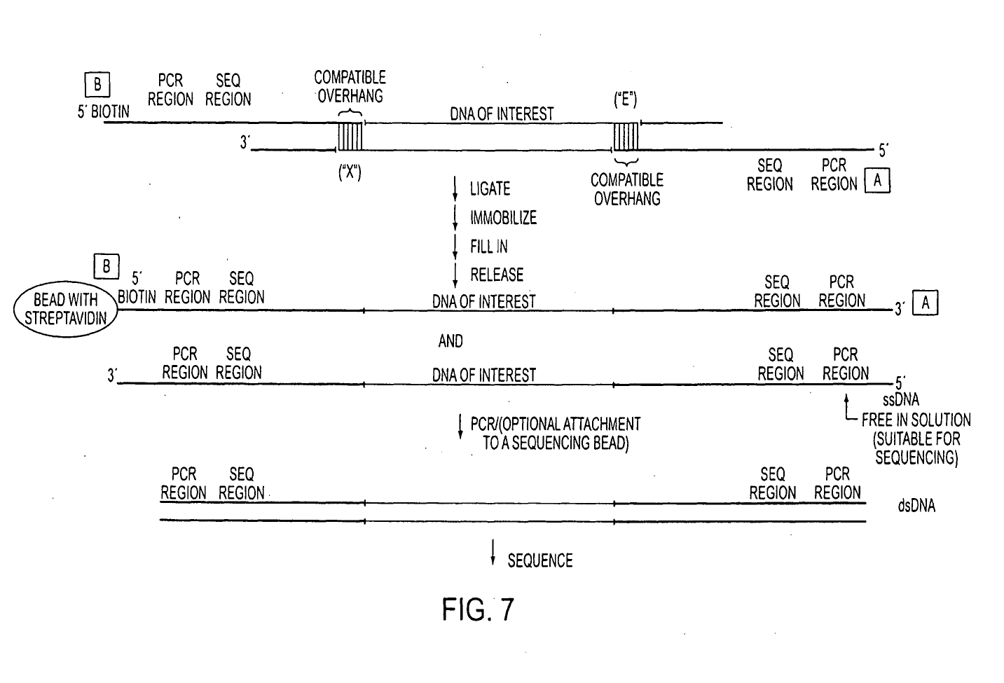

FIG. 8 diagrammatically illustrates the preparation of DNA molecules that are suitable for use in a sequencing method using the Applied Biosystems SOLiD™ sequencing technology.

### DESCRIPTION OF THE INVENTION

The present invention relates, e.g., to reagents and methods for isolating DNA molecules of interest in a form that is suitable for further analysis (e.g. for sequencing at least a portion of the DNA, for example by using a rapid, high throughput DNA sequencing method and apparatus). In methods of the invention, the DNA molecules of interest are flanked by products of restriction enzyme digestion, at least one of which has a sticky end. In one embodiment, the DNA molecules of interest are from accessible regions of chromatin (e.g., regulatory regions, such as transcriptionally active regions).

In one embodiment of the invention, DNA molecules containing regulatory sequences are isolated by a process comprising digestion of accessible regions of chromatin with at least two different restriction enzymes that generate single-strand overhangs (sticky ends); the digested DNA is converted by a method of the invention to a form that is suitable for sequencing in a high throughput sequencing procedure; and the DNA is sequenced with a conventional high throughput sequencing procedure. One inventive feature of the present invention is the use of defined adaptor molecules, each of which comprises a sticky end that is compatible with one of the sticky ends generated by the restriction enzyme digestion. The adaptors also comprise other sequences and/or elements (such as attachment agents) that allow the DNA to be sequenced in a high throughput apparatus. The adaptors can be modifications of conventional adaptors used for particular high throughput sequencing methods, except the blunt ends of the conventional adaptors are substituted with sticky ends that are compatible with the sticky ends of a DNA of interest to be sequenced. The adaptors are ligated to the digested DNA molecules via the compatible cohesive ends; and then DNA molecules containing the regulatory sequences, and flanked by the two adaptors, are isolated in a form suitable for further analysis, such as a high throughput sequencing procedure.

A method of the invention can be adapted for sequencing with any high throughput sequencing method. Typical such methods which are described herein include the sequencing technology and analytical instrumentation offered by Roche 454 Life Sciences™, Branford, Conn., which is sometimes referred to herein as “454 technology” or “454 sequencing.”; the sequencing technology and analytical instrumentation offered by Illumina, Inc, San Diego, Calif. (their Solexa Sequencing technology is sometimes referred to herein as the “Solexa method” or “Solexa technology”); or the sequencing technology and analytical instrumentation offered by ABI, Applied Biosystems, Indianapolis, Ind., which is sometimes referred to herein as the ABI-SOLiD™ platform or methodology.

Advantages of a method of the invention include that, when isolating accessible DNA fragments from chromatin, digestion by specific restriction enzymes rather than by non-sequence-specific nucleases or by shearing of the DNA circumvents the problem of background, e.g. resulting from cleavage of non-accessible DNA that is bound to histones, or from DNAs liberated due to random shearing or to single enzyme activity. This results in a high signal to noise ratio. Another advantage of digesting DNA with restriction enzymes rather than randomly shearing it is that the former procedure allows one to target and sequence regions of interest that lie near defined restriction enzyme sites. A method of the invention allows for the efficient, high-throughput, massively parallel isolation, identification and/or characterization (e.g. by sequencing) of regions (e.g., cis-acting transcriptional regulatory regions) in eukaryotic or other cells, and for the identification of putative target genes for these elements. Using a method of the invention, one can isolate and sequence, in parallel, a collection of all or nearly all of the regulatory sequences of, for example, a eukaryotic cell of interest. In methods of the invention, the DNA molecules can be isolated without having to clone/passage the DNA through a bacterium or other cell. This is advantageous for isolating and characterizing DNA molecules that are unstable or otherwise resistant to in vivo cloning.

One aspect of the invention is a method for isolating a DNA molecule of interest in a form that is suitable for sequencing at least a portion of the DNA by a high throughput sequencing method. The method comprises

digesting double-stranded (ds)DNA with two different restriction enzymes, A and B, that produce, as cleavage products, single-stranded overhangs (sticky ends), to generate a ds form of the DNA molecule of interest that is bounded by the two restriction enzyme cleavage products, and

attaching to each end of the DNA molecule of interest an adaptor molecule which comprises at one end a sticky end that is compatible with either the restriction enzyme A cleavage product or the restriction enzyme B cleavage product (sometimes referred to herein as “compatible cohesive ends”), and which also comprises one or more sequences and/or elements that allow the DNA of interest to be sequenced with a high throughput sequencing apparatus.

The two different restriction enzymes, A and B, generally produce cleavage products whose sticky ends are incompatible with one another. In some embodiments of the invention, “restriction enzyme A” refers to a collection (cocktail) of restriction enzymes (e.g., 2, 3 or more restriction enzymes), which generally have different, incompatible sticky-ended cleavage products. In sonic embodiments of the invention, the dsDNA can be digested with a single restriction enzyme.

The method can further comprise converting the ds form of the DNA molecule of interest, which is flanked by the adaptors, to a single-stranded (ss) form of the DNA; amplifying the ssDNA; and sequencing the amplified DNA with a high throughput sequencing apparatus.

The method can be adapted for sequencing with any of a variety of high throughput sequencing devices. The “sequences and/or elements” that are part of the adaptors and that allow the DNA of interest to be sequenced will vary according to which high throughput sequencing apparatus is to be used. In some instances, adaptors which have been employed to sequence blunt ended DNA with a particular apparatus are modified by a method of the invention to be used with restriction enzyme-digested DNA.

In one aspect of the invention, the high throughput sequencing apparatus used is a 454 instrument and the sequencing method is a modification of conventional 454 technology, wherein instead of the conventional adaptor used for 454 technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product.

For example, in one embodiment, after the adaptors have been added to the ds DNA of interest,

the ds form of the DNA of interest is bound to a surface (e.g. a magnetic bead coated with streptavidin) via an attachment agent (e.g. biotin) that is present at the end of one of the adaptors;

the bound, ds-DNA of interest is melted and single-stranded molecules of the DNA of interest are released from the surface and collected;

the released ssDNA is bound to a capture bead, via a sequence that is present in one of the adaptors, under conditions such that no more than one ssDNA molecule is attached to each bead;

the bound ss DNA is amplified by PCR, via a PCR priming site that is present in one of the adaptors; and

the amplified DNA is sequenced, via a sequence priming region that is part of one of the adaptors, using 454 technology.

In another aspect of the invention, the high throughput sequencing apparatus is a Solexa instrument, and the sequencing method is a modification of conventional Solexa technology, wherein instead of the conventional adaptor used for Solexa technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product.

For example, in one embodiment, after the adaptors have been added to the ds DNA of interest,

the dsDNA of interest is amplified by PCR to increase its copy number;

the amplified DNA is denatured to form single strands, the single strands are diluted, and single copies of the single-stranded form of the DNA of interest are bound, via a sequence that is present in one of the adaptors, to one of a plurality of oligonucleotides located at definable positions on a surface, under conditions such that no more than one DNA molecule is bound at each position on the surface;

the bound ssDNA molecule is amplified by bridge amplification, using sequences that are present in the adaptors, to form a clonal cluster on the surface; and

the bound, amplified form of the DNA in the clusters is sequenced, via a sequence priming region that is part of one of the adaptors, using Solexa technology.

In another aspect of the invention, the high throughput sequencing apparatus is an ABI instrument, the sequencing method is a modification of the conventional SOLiD™ method, wherein instead of the conventional adaptor used for the SOLiD™ technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product

For example, in one embodiment, after the adaptors have been added to the ds-DNA of interest,

the ds-DNA of interest is circularized by ligating each end of the DNA of interest to a DNA segment (sometimes referred to as an “internal adaptor”), wherein a sequence at the free end of each of the adaptors is compatible with a sequence at one of the ends of the DNA segment;

the circularized DNA is contacted with (treated with) the restriction enzyme EcoP151, under conditions such that the restriction enzyme binds to a recognition sequence that is present in each adaptor, and cuts downstream at a distance within the DNA of interest, to generate a linear double-stranded molecule that comprises, starting at one end of the linear molecule, about 25 bp from one end of the DNA of interest, the first adaptor, the DNA segment, the second adaptor, and about 25 bp from the other end of the DNA of interest;

the double-stranded linear molecule is ligated, at each end, to a molecule which comprises a PCR priming site, and the resulting dsDNA is amplified by PCR to increase its copy number;

the amplified DNA is denatured to form single strands, the single strands are diluted, and single copies of the single-stranded form of the DNA of interest are bound, via a sequence that is present in one of the adaptors, to a capture bead;

the bound ssDNA is amplified by PCR, via a PCR priming site that is present in one of the adaptors; and

the amplified DNA is sequenced, via a sequence priming region that is part of one of the adaptors, using ABI SOLiD™ technology.

In any of these methods, the DNA of interest may be from an accessible region of chromatin, e.g., an accessible region of chromatin which comprises regulatory and/or transcriptionally active sequences.

Much of the discussion herein is directed to embodiments of the invention in which DNA molecules are prepared so as to be suitable for sequencing in a 454 instrument. However, it is to be understood that aspects of this method can be readily adapted or modified for sequencing with other types of high throughput sequence devices.

One embodiment of the invention, which is directed to isolating a DNA molecule of interest that is suitable for sequencing at least a portion of the DNA with a 454 instrument, comprises

a) ligating to each end of a double-stranded (ds) form of the DNA molecule, which was generated by digestion with two restriction enzymes that produce sticky ends, an adaptor that comprises, in the following order, from the 5′ end of the molecule, a PCR primer region, a sequencing primer region, and a cohesive end that is compatible with one of the sticky ends, wherein one of the adaptors further has, at its 5′ end, an attachment agent (e.g. biotin),

b) binding the ligated DNA molecule to a surface (e.g. a bead, for example a bead that comprises streptavidin on its surface) via the attachment agent,

c) removing (separating) unbound DNA molecules,

d) treating the bound DNA molecule to fill in single-stranded regions (e.g. with T4 DNA polymerase), thereby forming a full-length dsDNA molecule; and

e) melting (separating) the strands of the fully dsDNA molecule, to release from the beads the single strand of the DNA molecule that lacks the attachment agent, and thus is not bound to the surface. Optionally, the released ssDNA can be captured for further analysis.

As used herein, the singular forms “a,” “an” and “the” include plural referents unless the context clearly dictates otherwise. For example, a method for isolating “a” DNA molecule, as used above, includes isolating a plurality of molecules (e.g. 10's, 100's, 1,000's, 10's of thousands, 100's of thousands, millions, or more molecules).

A “sticky end,” as used herein, refers to a configuration of DNA resulting, e.g., from the digestion of a double-stranded (ds)DNA with certain restriction enzymes. In this configuration, one strand of the DNA extends beyond the complementary region of the dsDNA, to possess a single-strand overhang. The single strand overhang may be a 5′ or a 3′ overhang. The single strand overhang can form complementary base pairs with the sticky end of another DNA molecule (e.g. cut with the same restriction enzyme, or with a compatible restriction enzyme that produces a complementary sticky end). The two single-stranded overhangs (sticky ends) are sometimes referred to as “compatible cohesive ends.” Two such fragments may be joined (covalently bonded) by a DNA ligase (sometimes referred to herein as a “ligase.”) A sticky end differs from a blunt end, in which the two DNA strands are of equal length, and thus do not terminate in a single-stranded overhang.

A DNA molecule that is “in a form suitable for sequencing,” as used herein, refers to a DNA molecule that, without further manipulation, can be sequenced. For example, in an embodiment of the invention directed to use with a 454 instrument, the DNA molecule “in a form suitable for sequencing” is a single-stranded DNA molecule which comprises, in the following order, starting from the 5′ end, an amplification region (e.g. a PCR priming region) and a sequence priming region.

The length of the “portion” of the DNA that is sequenced is a function of the amount of sequence information required for further analysis, and the sequencing method that is used. For example, for some forms of sequencing, such as a Solexa or the ABI SOLiD™ methods, about 20-30 nt from each end of the DNA of interest is sequenced; for other methods, such as a 454 method, at least about 230 nt from one or both ends can generally be sequenced. These and other methods for sequencing DNA are discussed further below.

In general, the order in which the steps of a method of the invention are performed is not critical; the steps can be performed in any order, or simultaneously. For example, in the preceding method using the 454 instrument, the adaptors may be ligated to the dsDNA molecule before or simultaneously with the binding of the DNA to the surface. In embodiments of the invention, the adaptors, DNA of interest, ligase, and surface may all present together in a reaction mixture; or the DNA may be ligated first to the adaptors, then bound to the surface. In another example, the step to “fill-in” the single-stranded regions may be performed after the DNA has been ligated to the adaptors but before it is bound to the surface; after the DNA has been bound to the surface, but before unbound DNA molecules have been removed (a wash step); or after the wash step. In a preferred embodiment, the “fill-in” step is performed after the DNA has been immobilized to the surface and undesired DNA molecules have been washed away, and before the melting step. By washing away undesired DNA fragments before the fill-in reaction takes place, the DNA polymerase does not have to fill in the undesired fragments, and thus may be more efficient than if the undesired DNA were present. In some embodiments, it may be desirable to centrifuge down beads containing bound DNA, or in the case of magnetic beads, to remove them with a magnet (probe), in order to change the local environment of the DNA. For example, one can change the buffer to an optimal buffer for treatment with an enzyme (e.g. ligase or DNA polymerase); or one can introduce conditions for melting (separating) the strands of a dsDNA molecule, such as contacting the dsDNA with a basic solution. As used herein, the term to “melt” the strands of a dsDNA is used interchangeably with the term to “separate” the strands.

Another aspect of the invention is a method as above, which is adapted for sequencing with a 454 apparatus, wherein the dsDNA molecule of interest is flanked at one end with sequence A, which is a digestion product of restriction enzyme A, and at the other end by sequence B, which is a digestion product of restriction enzyme B. At least one of restriction enzyme A or restriction enzyme B produces a sticky end, which can have either a 5′ or a 3′ overhang. In one embodiment, both of the enzymes (or collections of enzymes, such as a cocktail of enzymes) produce sticky ends. The method comprises

a) contacting the double-stranded form of the DNA molecule (dsDNA) with two adaptors:
i) a first partially duplex adaptor, adaptor A, which comprises, in the 5′ to 3′ direction, in the following order, a single-stranded portion comprising a PCR priming region and a sequence priming region, and then a double-stranded portion with a single-stranded overhang that is compatible with the digestion product of restriction enzyme A, and
ii) a second partially duplex adaptor, adaptor B, which comprises, starting at the 5′ end, an attachment agent (e.g. biotin), a single-stranded portion comprising a PCR priming region, a single-stranded sequence priming region, and a double-stranded portion with a single-stranded overhang that is compatible with the digestion product of restriction enzyme B,

under conditions that are effective to join the dsDNA molecule to the two adaptors (by annealing the complementary single-stranded overhangs of the compatible digestion products), to ligate nicks thus formed (e.g. with T4 DNA ligase), and to attach the joined ligated, partially dsDNA molecule to a surface, thereby obtaining a joined ligated, partially dsDNA molecule which is attached to the surface;

b) separating the joined partially dsDNA molecule attached to the surface from unbound DNA molecules; and

c) subjecting the joined partially dsDNA molecule attached to the surface to conditions effective for tilling in single-stranded regions, separating strands of the DNA molecule bound to the surface, and removing from the surface the single-full-length strand of the DNA which lacks the attachment agent, thereby isolating a single-stranded DNA molecule comprising the sequence of the DNA of interest, in a form suitable for sequencing at least a portion of the DNA of interest.

Another aspect of the invention is a method for sequencing regulatory elements within a cell, comprising

subjecting a collection of dsDNA molecules that are enriched for regulatory elements and are also flanked by digestion products (with sticky ends) of restriction enzymes A and B to a method of the invention for isolating a DNA molecule, thereby isolating a collection of single-stranded DNA molecules comprising the regulatory elements in a form suitable for sequencing at least a portion of each of the DNA molecules, and

sequencing at least a portion of each of the DNA molecules.

Other aspects of the invention include adaptors used in a method of the invention and kits comprising those adaptors.

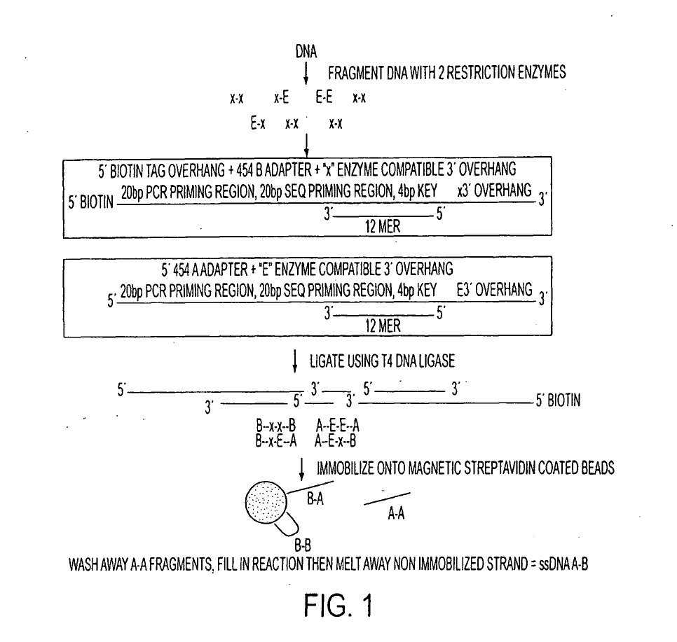

By way of example, FIG. 1 illustrates schematically one embodiment of the invention. In this figure, a collection of DNA molecules is generated by digesting a larger DNA molecule with two restriction enzymes, E and x. (In one embodiment of the invention, which is illustrated in Example 1, enzyme E is NlaIII, and enzyme x is Sau3A I.) The desired products are the double-stranded (ds)DNA fragments that are flanked at one end by the digestion product of restriction enzyme E and at the other end by the digestion product of restriction enzyme x (referred to in the figure as “E-x” or “x-E”). Other, undesired, DNA molecules will also be generated, which are flanked by restriction enzyme cuts by x alone (“x-x”) or E alone (“E-E”). The mixture of digested DNAs is ligated to two partially duplex adaptor molecules—A and B—which are shown in the figure. Note that one of the adaptors—adaptor B—has, at its 5′ end, an attachment agent (in this case, biotin). Four types of ligated molecules are formed: the desirable B-x-E-A and A-E-x-B molecules, and the undesired molecules B-x-x-B and A-E-E-A.

The mixture of four types of ligated molecules is contacted with a surface (in this case, magnetic beads coated with streptavidin). Molecules A-E-E-A, which lack biotin, do not bind to the beads, and thus can be readily washed away. The desired molecules, B-x-E-A and A-E-x-B, bind to the beads via the DNA strand in each duplex that contains the 5′ biotin. Molecules B-x-x-B bind to the beads, such that each of the two strands in the duplex is bound via the biotin molecule at its 5′ end.

The bound DNA molecules are then treated under conditions effective for removing from the surface (and thereby isolating) the desired single-stranded, full-length molecules flanked by digestion products of restriction enzymes x and E. The effective conditions can support the following reactions: The ligated molecules are treated with a DNA polymerase, such as T4 DNA polymerase, which fills in the single-stranded regions in each of the molecules (see FIG. 1), thereby generating full-length strands of DNA for each strand of the duplex. The dsDNA molecules bound to the beads are then melted apart. In the case of the B-x-x-B dsDNA molecules, both strands will remain bound to the beads via the biotins at their 5′ ends. However, in the case of the B-x-E-A and A-E-x-B dsDNA molecules, the strand of the duplex that is labeled with a biotin will remain bound to the beads, but the strand that does not contain a biotin will be melted off and released from the bead. The released single strands may then be collected (e.g. by removing the magnetic beads carrying undesired DNA molecules). This process results in the isolation of full-length single-stranded DNA molecules of interest that are flanked by different restriction enzyme digestion products.

In variations of the illustrated method, the treatment with DNA polymerase (a “fill-in” reaction) is performed after the ligation step, but before the DNA molecules are bound to the beads; before undesired A-E-E-A molecules are washed away; or after they have been washed away, but before the melting step is carried out. It is sometimes desirable to bind the ligated DNA molecules to the beads, to separate the beads carrying the ligated DNA from the solution, and to replace the solution with a buffer more compatible with subsequent reactions, before treating the DNA under conditions for DNA polymerase to till in single-stranded regions.

The isolated collection of sequences may be analyzed in any of a variety of ways, e.g. by sequencing portions of the DNA fragments.

In one embodiment of the invention, a collection of dsDNA fragments that are highly enriched for regulatory sequences is generated such that each fragment is flanked by different restriction enzyme digestion products; and single-stranded molecules which are in a form suitable for further analysis are isolated by a method of the invention. In one embodiment, the collection of dsDNA molecules is generated as follows: Chromatin from genomic DNA (from a cell's nucleus) is digested by a cocktail of multiple (e.g. three) restriction enzymes (“A”) with different sequence specificities (e.g. HpaII, MseI and NlaIII) that digest “accessible” regions in the chromatin; the digested chromatin is then deproteinized; and the deproteinized DNA is digested with a restriction enzyme (“B”) that cuts often in the DNA, such as a “4-cutter” (e.g. Sau3A I). The DNAs in this collection of digested DNA molecules, which are enriched for accessible (e.g. regulatory, including transcriptionally active) sequences, are then optionally size fractionated to obtain DNA fragments suitable for DNA amplification and/or sequencing (e.g. about 100-400 bp), and are treated by a method of the invention to isolate a collection of single-stranded DNA molecules, flanked by the two restriction enzyme digestion products, that are enriched for regulatory sequences. With this embodiment of the invention, an investigator can obtain at least about 94% of the regulatory elements of a cell of interest.

A method of the invention can be used to isolate and, optionally, characterize (e.g. by sequencing) any DNA of interest (including collections of many such DNA molecules) that is flanked by two different restriction enzyme cleavage sites. The ends of nucleic acids resulting from digestion by a restriction enzyme at a restriction enzyme recognition site (cleavage site, recognition sequence) are sometimes referred to herein as “products of digestion by a restriction enzyme.” Preferably, restriction enzymes used in methods of the invention produce sticky ends, with either 5′ or 3′ single-strand overhangs. The product of digestion by a restriction enzyme can be ligated to a DNA whose end is “compatible” with that digestion product. In general, two products of restriction enzyme digestion are compatible if the single-stranded overhangs generated by the digestion are complementary and can be annealed specifically to one another (compatible cohesive ends). The two DNAs can then be ligated. Examples of compatible ends include: ends generated by digestion with the same restriction enzyme; and ends digested by different restriction enzymes, such as HpaII and ClaI, Sau3A I and BamHI, or NlaIII and SphI. Other suitable pairs of restriction enzymes will be evident to the skilled worker. When sticky ends generated by two different restriction enzymes are joined, the resulting sequence is sometimes referred to herein as a “composite sequence.”

Methods of carrying out the techniques used in methods of the invention will be evident to the skilled worker. For example, conventional methods (e.g., chemical synthesis and/or digestion of DNA with restriction enzymes) can be employed to generate the modified adaptors of the invention. The practice of conventional techniques in molecular biology, biochemistry, chromatin structure and analysis, computational chemistry, cell culture, recombinant DNA, bioinformatics, genomics and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N.Y., 1989; Ausubel et al., Current Protocols in Molecular Biology, John Wiley & Sons, New York, 1987 and periodic updates; the series Methods in Enzymology, Academic Press, San Diego; Wolffe, Chromatin Structure and Function, Third edition, Academic Press, San Diego, 1998; Methods in Enzymology, Vol. 304, “Chromatin” (P. M. Wassarman and A. P. Wolffe, eds.), Academic Press, San Diego, 1999; and Methods in Molecular Biology, Vol. 119, “Chromatin Protocols” (P. B. Becker, ed.) Humana Press, Totowa, 1999.

The disclosed methods can be used to isolate and, optionally, sequence nucleic acid molecules from any source, including a cellular or tissue nucleic acid sample, a subclone of a previously cloned fragment, mRNA, chemically synthesized nucleic acid, genomic nucleic acid samples, nucleic acid molecules obtained from nucleic acid libraries, specific nucleic acid molecules, and mixtures of nucleic acid molecules. When digesting chromatin, whole cells, isolated nuclei, nuclear extracts, or bulk cellular DNA or chromatin can be used.

In one embodiment of the invention, a method is used to discover and characterize genetic variation in a set of human DNA samples. In this embodiment, naked, genomic DNA is digested with an “8-cutter,” “10-cutter,” or higher restriction enzyme (e.g. EcoO1091, NotI, AscI, BglI, or many others that will be evident to the skilled worker), followed by a “4-cutter,” such as Sau3A. Suitable restriction enzymes and digestion conditions are selected for identifying a reproducible set of regions for genome sequencing in a population of DNA samples. Following this double digestion, the resulting DNA fragments are treated as described below for the identification of regulatory regions (e.g. size fractionation to obtain DNA fragments of about 100-400 bp, followed by ligation to adaptors with suitable ends, etc.) For example, for DNA digested with EcoO1091 and Sau3A I, one can ligate the double digested DNA to adaptors with EcoO1091 and Sau3A I ends, respectively. This pair of enzymes allows one to reproducibly sequence about 1.3 million unique genomic regions, some 6% of which cover 36% of all exons in the human genome. A similar approach can be used to “re-sequence” DNA molecules, to independently confirm previous sequencing of the DNA.

In another embodiment of the invention, regions of DNA that are “accessible” in chromatin (e.g., regulatory regions, such as transcriptionally active portions of DNA) are isolated and, optionally, sequenced.

Chromatin is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin. A chromosome is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

“Accessible” regions of chromatin are regions that can be contacted more efficiently by agents, such as chemical probes or enzymes that cleave DNA, than are other regions in cellular chromatin. Accessibility is any property that distinguishes a particular region of DNA, in cellular chromatin, from bulk cellular DNA. For example, an accessible sequence (or accessible region) can be one that is not packaged into nucleosomes, or can comprise DNA present in nucleosomal strictures that are different from that of bulk nucleosomal DNA (e.g., nucleosomes comprising modified histones). An accessible region includes, but is not limited to, a site in chromatin at which a restriction enzyme can cut, under conditions in which the enzyme does not cut similar sites in bulk chromatin. Accessible regions include, e.g., a variety of cis-acting, regulatory elements. Regulatory sequences are estimated to occupy between 1 and 10% of the human genome. Such regulatory elements can be present both within and flanking coding sequences. Among such regulatory regions are, e.g., promoters, enhancers, silencers, locus control regions, boundary elements (e.g., insulators), splice sites, transcription termination sites, polyA addition sites, matrix attachment regions, sites involved in control of replication (e.g., replication origins), centromeres, telomeres, and sites regulating chromosome structure.

A variety of methods can be used to digest chromatin to obtain accessible (e.g. regulatory) regions. The methods disclosed herein allow the identification, isolation (e.g. purification) and characterization (e.g. sequencing) of regulatory sequences in a cell of interest, without requiring knowledge of the functional properties of the sequences.

One way to identify accessible DNA is by selective or limited cleavage of cellular chromatin to obtain polynucleotide fragments that are enriched in regulatory sequences. One approach is to perform limited digestion of whole cells, isolated nuclei or bulk chromatin with a restriction enzyme (restriction endonuclease) or a collection of restriction enzymes under conditions for cutting about one time in each accessible region, preferably no more than one time in each region. Generally, a brief exposure to the enzyme(s) is sufficient; the digestion conditions can be determined empirically. Because the digestion with this first restriction enzyme(s) (sometimes referred to herein as “restriction enzyme A”) is designed to produce only about one cut in each accessible region in chromatin, the resulting DNA fragments will be very long. To digest these fragments further, to render them a size more amenable to amplification and/or DNA sequencing, the DNA that has been digested with restriction enzyme(s) A is deproteinized (deproteinated), using a conventional procedure, and is then digested to completion with a secondary enzyme (sometimes referred to herein as “restriction enzyme B”), preferably one that has a four-nucleotide recognition sequence (a “4-cutter”), such as Sau3A I. Optionally, one can reduce random shearing of the long DNA molecules, which can generate artifactual ends, by embedding the DNA digested with restriction enzyme(s) A in an agarose (e.g. low melting agarose) plug. The secondary enzyme can then be diffused into the plug, where it digests the DNA.

Any of a variety of first restriction enzymes (restriction enzyme A or, as indicated in FIG. 1, restriction enzyme E) can be used.

In one embodiment of the invention, chromatin is digested with a restriction enzyme that cuts in sequences that are enriched in CpG islands. The dinucleotide CpG is severely underrepresented in mammalian genomes relative to its expected statistical occurrence frequency of 6.25%. In addition, the bulk of CpG residues in the genome are methylated (with the modification occurring at the 5-position of the cytosine base). As a consequence of these two phenomena, total human genomic DNA is remarkably resistant to, for example, the restriction endonuclease Hpa II, whose recognition sequence is CCGG, and whose activity is blocked by methylation of the second cytosine in the target site.

An important exception to the overall paucity of demethylated Hpa II sites in the genome are exceptionally CpG-rich sequences (so-called “CpG islands”) that occur in the vicinity of transcriptional start sites (e.g. in front of the approximately 40% of genes that are constitutively active, i.e. housekeeping genes), and which are demethylated in the promoters of active genes. Aberrant hypermethylation of such promoter-associated CpG islands is a well-established characteristic of the genome of malignant cells.

Accordingly, one option for cleaving within accessible regions relies on the observation that, whereas most CpG dinucleotides in the eukaryotic genome are methylated at the C5 position of the C residue, CpG dinucleotides within the CpG islands of active genes are unmethylated. (See, for example, Bird (1992) Cell 70, 5-8, and Robertson et al. (2000) Carcinogenesis 21, 461-467.) Indeed, methylation of CpG is one mechanism by which eukaryotic gene expression is repressed. Accordingly, a methylation-sensitive restriction enzyme (i.e., one that does not cleave methylated DNA), especially one with the dinucleotide CpG in its recognition sequence, such as, for example, Hpa II, will cleave cellular chromatin in the accessible regions of DNA. A variety of suitable enzymes will be evident to the skilled worker. For example, the 2005-6 catalogue from New England BioLabs, Inc., Beverly, Mass. (NEB) lists over 40 such enzymes, including HpaII and ClaI. Suitable enzymes for this, or other aspects of the invention, are available commercially, e.g. from NEB.

Other restriction enzymes can also be used to digest accessible regions of chromatin. Some of the Examples herein illustrate the use of NlaIII, a restriction enzyme whose recognition sequence, 5′ . . . CATG . . . 3″, falls into the class of sequences that consist of a palindromic combination of A, G, C and T residues. A large number of suitable restriction enzymes in this category will be evident to the skilled worker. Preferably, to maximize the number of cuts, the enzyme is a 4-cutter.

Another class of restriction enzymes that can be used are enzymes that cut in A-T-rich sequences, particularly sequences that consist solely of A's and T's. Many such enzymes having this property are available, e.g. MseI and Tsp5091.

In one embodiment, a cocktail (combination) comprising multiple (e.g. 2, 3, 4, 5 or more, preferably 3) restriction enzymes is used to digest accessible regions in chromatin. In order to maximize the number of cleavages in accessible regions, a cocktail of enzymes having different sequence specificities is used. For example, the cocktail may contain HpaII, NlaIII and MseI. In order to facilitate ligation with the digested DNA to the adaptors of the invention, restriction enzymes that leave sticky ends (with either 5′ or 3′ overhangs) are preferred.

Thus, in one method of the invention, one or more restrictions enzymes are used to digest accessible regions of chromatin e.g. regulatory regions, such as in transcriptionally active DNA. The restriction enzyme, sometimes referred to herein as restriction enzyme A, can comprise, e.g.,

a) a methylation-sensitive enzyme that contains a CG dinucleotide in its recognition sequence (e.g., that cleaves unmethylated CG-containing sites in CpG islands). One representative of such as enzyme is HpaII;

b) an enzyme that cuts sequences having solely A or residues (e.g., MseI); and/or

c) an enzyme whose recognition site consists of a palindromic combination of A, G, C and T (e.g., NlaIII).

Preferably, the restriction enzyme(s) produce sticky ends after digestion (either 3′ or 5′ overhangs).

In embodiments of the invention, restriction enzyme A is a combination (cocktail) comprising at least one of HpaII, MseI, or NlaIII. Restriction enzyme A may be a combination comprising two of HpaII, MseI, and NlaIII or comprising all three of HpaII, MseI, and NlaIII. In one embodiment, restriction enzyme A is a combination consisting of HpaII, MseI, and NlaIII.

In another embodiment of the invention, deproteinized genomic DNA is first digested with agents that selectively cleave AT-rich DNA. Examples of such agents include, e.g., restriction enzymes having recognition sequences consisting solely of A and T residues. Examples of suitable restriction enzymes include, but are not limited to, MseI, Tsp509 I, AseI, DraI, SspI, PacI, SwaI and PsiI. Because of the concentration of GC-rich sequences within CpG islands (see above), large fragments resulting from such digestion generally comprise CpG island regulatory sequences, especially when a restriction enzyme with a four-nucleotide recognition sequence consisting entirely of A and T residues (e.g., Mse I, Tsp509 I) is used as a digestion agent. Such large fragments can be separated, based on their size, from the smaller fragments generated from cleavage at regions rich in AT sequences. In certain cases, digestion with multiple enzymes recognizing AT-rich sequences provides greater enrichment for regulatory sequences. The digested DNA can them be digested further with a 4-cutter and ligated to suitable adaptors and subjected to an isolation method of the invention.

Any of a variety of secondary restriction enzymes can be used to digest the regulatory sequences into smaller fragments. The second restriction enzymes are sometimes referred to herein as restriction enzyme B (or, in FIG. 1, restriction enzyme x). Preferably, the secondary restriction enzyme recognizes a 4-base recognition sequence (cutting site) and results in a sticky end. The skilled worker will recognize a variety of suitable secondary enzymes (e.g. NlaIII or others). In some of the Examples herein, Sau3A I is used.

The double digested DNA fragments can be size fractionated, if desired, in order to obtain fragments that are optimal in length for amplification and/or DNA sequencing (for example, about 100-2000 bp (e.g. about 100-400 bp or about 800-2000 bp), depending on the sequencing procedure). Various separation methods can be used, including, e.g., gel electrophoresis, sedimentation and size-exclusion columns, or differential solubility. In one embodiment, agarose gel electrophoresis is used.

Other methods to isolate regulatory DNA that can be subjected to an isolation method of the invention will be evident to the skilled worker. Some such methods, including methods that involve methylating accessible sites in chromatin and isolating the DNA thus methylated, are described in U.S. Pat. No. 7,097,978.

In a method of the invention, particular adaptors are joined (ligated) to the compatible ends of the doubly digested DNA of interest. An adaptor of the invention can comprise, in the following order, starting from the 5′ end, an amplification region (e.g. a PCR priming region), a sequencing priming region, and a cohesive end that is compatible with one of the sticky ends of the DNA to be isolated. See FIG. 1 for an illustration of an adaptor of the invention.

Any conventional form of amplification can be used. Preferably, the amplification is PCR amplification, and the amplification region is a PCR priming region, which includes a sequence for a PCR primer (or the complement thereof). The sequencing priming region includes a sequence (or the complement thereof) of a primer for initiating DNA sequencing. The amplification and sequence priming regions allow the DNA of interest to be amplified to a sufficient level to be sequenced, and provides a site at which a sequencing primer can be bound for the initiation of DNA synthesis. The sequencing priming region is preferably adjacent or nearly adjacent to the restriction enzyme recognition sequence. Thus, the restriction enzyme sequence is the only extraneous sequence between the sequencing primer and the DNA of interest. Generally, the sequence primer regions in adaptor A and adaptor B are different, allowing the released ssDNA to be sequenced, independently, from either sequence primer (in either direction). In some embodiments, e.g. when a 454 apparatus is used to sequence the DNA of interest, a 4 base “key” sequence may also be present in the adaptor, 3′ to the sequence primer region. Software in the 454 Sequence apparatus rejects any sequences that do not contain this key sequence, as a quality control measure. In other embodiments, the presence of the restriction enzyme cutting site in a sequence confirms that the DNA being sequenced is, indeed, DNA that has been joined correctly to an adaptor of the invention.

When chromatin has been cut with a cocktail of restriction enzymes (e.g. with 3 enzymes), to create a mixture of fragments having different single-stranded overhangs at their ends, a mixture of adaptors, with ends compatible with the ends of the fragments in the mixture, are ligated to the mixture of DNA fragments. For example, if chromatin is cut with, as restriction enzyme A, HpaII. NlaIII and MseI, three different adaptor A molecules are included in the ligation mixture, having cohesive ends that are compatible with each of the three restriction enzyme digestion products.

Adaptors of the invention can be prepared by conventional methods. For example, the individual strands can be synthesized with a commercially available or custom-designed synthesizer, and then annealed to form the partially dsDNA molecule.

One of the two partially double-stranded (ds) adaptors that are ligated to each DNA molecule of interest comprises, at its 5′ end, an attachment agent. Any agent can be used which facilitates the attachment of the DNA on which it is located to a suitable surface. A variety of suitable attachment agents will be evident to the skilled worker, for attachment to any suitable surface. In one embodiment, the attachment agent is biotin, which reacts avidly and specifically with streptavidin. Methods for attaching a biotin molecule to the 5′ end of a DNA molecule are well-known and conventional.

The end of an adaptor of the invention having the biotin moiety is sometimes referred to herein as the “distal” end of the adaptor (distal to the dsDNA molecule of interest); the other end of the adaptor, having the end which is compatible with the restriction enzyme cut site of the DNA of interest, is sometimes referred to herein as the “proximal” end of the adaptor.

Following (or at substantially the same time as) the ligation of the adaptors to the DNA molecules of interest, the DNA molecules are bound (attached, immobilized) to a surface via the attachment agent. Any of a variety of suitable surfaces will be apparent to the skilled worker. These surfaces include, e.g., plastics such as polypropylene or polystyrene, ceramic, silicon, (fused) silica, quartz or glass (which can have the thickness of, for example, a glass microscope slide or a glass cover slip), paper, such as filter paper, diazotized cellulose, nitrocellulose, filters, nylon membrane, polyacrylamide gel pad, etc. In one embodiment of the invention, the attachment agent is biotin and the surface is a magnetic bead that is coated with avidin.

The double-stranded DNA molecules of interest are contacted with the adaptor molecules under conditions that are effective to join the DNA molecules to the adaptors (e.g. by annealing the complementary single-stranded overhangs), to ligate the nicks thus formed (e.g. with a ligase, such as T4 ligase), and to attach the joined, ligated, partially dsDNA molecule to the surface. The effective conditions can include, e.g., the presence of a suitable amount (e.g. in a reaction vessel, a reaction mixture, or the same solution) of the adaptors, the ligase, and the surface, and suitable additional reaction components, including buffers, salts, co-factors or the like.

As noted, any suitable attachment agent and surface can be used. The following discussion is directed to a combination of biotin and magnetic beads coated with streptavidin. However, any combination of attachment agent and surface is included. Following the attachment of DNA molecules bearing 5′ attachment agents (e.g. biotin) to magnetic beads, the beads can be separated from undesired molecules, such as components of a reaction mixture, by the use of a magnet or magnetized probe. For example, following immobilization of biotin-labeled DNA molecules of interest to beads comprising streptavidin on their surface, the beads can be washed to remove (to separate) undesired DNA molecules that do not bind to the beads. As indicated in FIG. 1, molecules having the structure A-E-E-A can be so removed.

In order to isolate the desired single-stranded DNA molecules comprising the DNA of interest, in a form suitable for further analysis, such as DNA sequencing, the joined, partially dsDNA molecules attached to the surface are subjected to conditions effective for separating the strands of the DNA molecule bound to the surface and for removing from the surface the single-strand, hill-length strand of the DNA which lacks the binding partner. The effective conditions allow for the following steps to take place: filling in the single-stranded portions of the joined, partially dsDNA, to form dsDNA (if this step has not already been performed); treating the dsDNA under effective conditions to separate (melt) the strands of the dsDNA (e.g. contacting the DNA with 0.125N NaOH); and separating the released single-stranded DNA strand which lacks the binding partner. For example, the effective conditions may comprise the presence of a suitable amount (e.g. in a reaction vessel, in a reaction mixture, or the same solution) of an enzyme, such as T4 DNA polymerase, and suitable additional reaction components, including buffers, salts, co-factors or the like, for filling in the single-stranded portions of the joined, partially dsDNA, to form dsDNA; and (optionally in a subsequent step) sufficient heat and/or chemical agents (e.g. basic conditions) to melt (separate) the strands of the dsDNA.

Optionally, the released ssDNA can be collected.

Following isolation of the desired ssDNA molecules, at least a portion of each of the ssDNAs may be amplified, in order to generate a sufficient quantity to be sequenced. Any suitable amplification method may be used. In a preferred embodiment, the amplification is PCR amplification, using primers that correspond to (are complementary to, or have the same sequence as) PCR amplification regions in adaptors A and B. In one embodiment, amplification is carried out by emulsion PCR (emPCR). The size of the DNA that must be amplified is dependent on the subsequent steps to be carried out on the DNA. For example, if the DNA is to be sequenced, it is desirable to amplify the entire DNA of interest.

Any of a variety of well-known, conventional methods can be used to sequence the DNA molecules isolated by a method of the invention. Generally, it is only necessary to sequence about 20-50 bases from one end: the end that was digested from accessible chromatin (e.g., the NlaIII end) of a DNA molecule of interest (in addition to the restriction enzyme recognition site), because this is the portion of the DNA that is truly accessible and thus potentially regulatory. If desired, the DNA can also be sequenced from the end generated by the secondary restriction enzyme (e.g. Sau3A I), to confirm and/or extend the first sequence. In general, digestion with only a single “secondary” restriction enzyme allows about 2-3 fold coverage of a mammalian genome if between about 30,000-50,000 sequences are determined.

One sequencing method that can be used on single-stranded DNA molecules isolated by a method of the invention is a modification of the 454 method (e.g., using the modified adaptors of the invention, which have sticky end restriction enzyme sites at one end). This method uses a 454 Genome Sequencer 20 or FLX (454 Life Sciences, Roche Applied Sciences). See, e.g., Margulies et al. (2005) Nature 437, 376-80; Rogers et al. (2005) Nature 437, 326-7; or the technical manual available on the web site for 454 Life Sciences. See also the patent application assigned to the 454 company, US2005/0079510. Such devices have extremely high throughput. Generally, between about 80 and about 130 bases are sequenced with the Genome Sequencer 20 apparatus, or between about 200 and 250 bases with the FLX apparatus. An accurate read of about 100 bases is currently claimed by the 454 Life Sciences company for the Genome Sequencer 20 apparatus, and an accurate read of about 230 is claimed by the current version of the machine, the FLX apparatus. Suitable reagents for carrying out the sequence reactions can be purchased from commercial suppliers, such as Roche Applied Biosciences (Indianapolis, Ind.).

In one embodiment of the invention, the released single-stranded DNA is quantitated by a conventional method (e.g. by using an RNA Pico 6000 LabChip) and diluted appropriately, then attached to a bead, such as a 454 capture bead (a sepharose bead), so that only one ssDNA molecule is attached to each bead. The capture bead may comprise (e.g. be coated by) a capture primer that is complementary to a sequence present in the adaptor molecule. The capture primer essentially provides an anchor to which the single-stranded molecule can hybridize. See, e.g., US2005/0079510 for details of such a process. When sequencing DNA from an accessible region that has been cut with restriction enzyme A, it is generally preferable that the capture primer hybridizes to a sequence in the B adaptor; this leaves the A adaptor end free for pyrosequencing to begin from that end. In contrast, if it is desired to sequence the released ssDNA in the opposite direction, the capture primer preferably hybridizes to a sequence in the A adaptor; this leaves the B adaptor end free for sequencing to begin from that end. The DNA is then amplified (e.g. using emPCR), and at least about 100 bases (using the Gene Sequencer 20 apparatus) or at least about 230 bases (using the FLX apparatus) from the amplified DNA molecule is sequenced, e.g. using a 454 sequencing system.

Another sequencing method that can be employed is a modification of the conventional Solexa Sequencing technology (offered by Illumina). The modification substitutes the modified adaptors of the invention, which have sticky end restriction enzyme cleavage products at one end, for the conventional adaptors. Sequencing with this device involves bridge amplification on a solid surface, as described, e.g., on the web site for the Promega company and the web site for Illumina (Solexa). Bridge amplification employs primers bound to a solid surface for the extension and amplification of solution phase target nucleic acid sequences. The term “bridge amplification” refers to the fact that, during the annealing step, the extension product from one bound primer forms a bridge to the other bound primer. All amplified products are covalently bound to the surface. Because the Solexa sequencing method involves an A and a B primer, DNA molecules ligated to adaptors A and B of the invention can also be sequenced by this method. Conventional procedures for Using this apparatus are well known in the art, and are available from the manufacturer. In general, sequencing with the Solexa sequencing method is not directional, so portions of both ends of a DNA molecule of interest are generally sequenced. The method may be adapted to allow sequencing from one end of particular interest.

Another sequencing method that can be used is a modification of the conventional sequencing method utilizing a the Applied Biosystems SOLiD™ sequence technology (from Roche Applied Biosciences, Indianapolis, Ind.). The modification substitutes the modified adaptors of the invention, which have sticky end restriction enzyme cleavage products at one end, for the conventional adaptors. The Applied Biosystems SOLiD™ System is a genetic analysis platform that enables massively parallel sequencing of clonally amplified DNA fragments linked to magnetic beads. The sequencing methodology is based on sequential ligation with dye-labeled oligonucleotides. In this method, the DNA sequence is generated by measuring the serial ligation of an oligonucleotide by ligase. All fluorescently labeled oligonucleotide probes are present simultaneously and compete for incorporation. After each ligation, the fluorescence signal is measured and then cleaved before another round of ligation takes place.

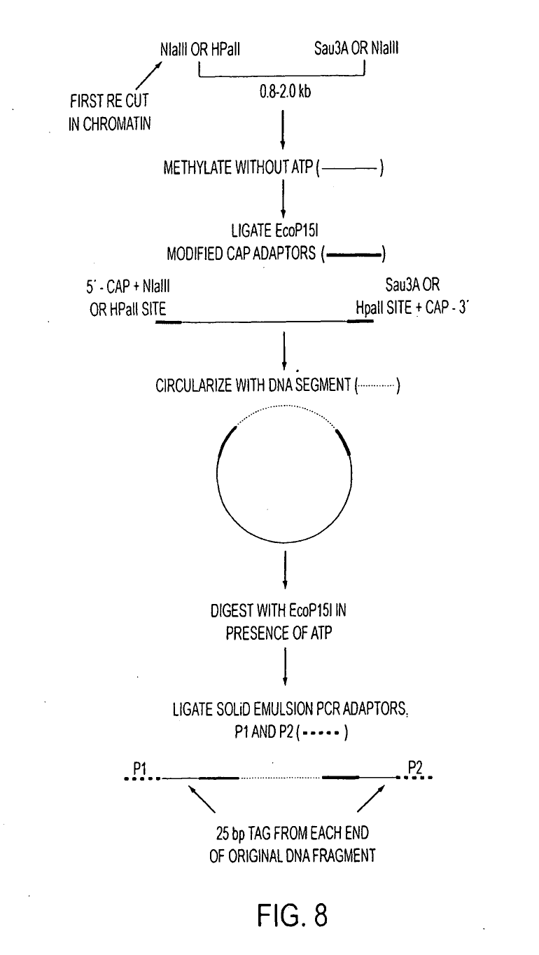

This enables the sequencing platform to generate sequence reads of up to 35 bp in length targeting about 125 million clone ends per run producing about 1.6 Gbases of usable sequence. This platform is ideal for screening the full cis-regulatory component of a cell's DNA in a single run. The modified sample preparation procedure needed to screen restriction fragments produced from a chromatin preparation (or from any other source of interest) is outlined in FIG. 8. In general, sequencing with the ABI SOLiD™ method is not directional, so portions of both ends of a DNA molecule of interest are generally sequenced. The method may be adapted to allow sequencing from one end of particular interest.

As shown in FIG. 8, following digestion of DNA (e.g. from chromatin) with restriction enzyme A (e.g. NlaIII or HpaII) and restriction enzyme B (e.g. Sau3A or NlaIII) and, if desired, the isolation of doubly digested fragments of about 0.8-2.0 kb, the DNA is methylated without ATP to protect EcoP151 recognition sites; and modified CAP linkers, which contain overhangs compatible with restriction enzyme A or restriction enzyme B cleavage products, and which contain EcoP151 recognition sites, are ligated to the DNA fragments via the restriction enzyme A and B cut sites. These ligated DNA molecules are then circularized, using a DNA segment with suitable compatible sticky ends. The circularized DNA is then digested with EcoP151 in the presence of ATP. The enzyme binds at the EcoP151 recognition sites in the adaptors, but cuts downstream at a distance (about 25 bp) in the DNA of interest (indicated in the figure as a solid line). The linear molecule is then ligated to SOLiD™ emulsion PCR adaptors and processed by conventional SOLiD™ procedures. For the purposes of illustration, EcoP151 is used, but it will be evident to a skilled worker that equivalent restriction enzymes, which also cut downstream at a distance, can be substituted for EcoP151.

More details of the SOLiD™ methodology can be found, e.g., at the world wide web site: http://marketing.appliedbiosystems.com/mk/get/SOLID_KNOWLEDGE_LANDING?_A=80414&_D=52611&_V=0. In general, sequencing with the SOLiD™ sequencing technology is not directional, so portions of both ends of a DNA molecule of interest are generally sequenced.

Thus, one aspect of the invention is a method for sequencing regulatory elements within a cell, comprising

subjecting a collection of dsDNA molecules that are enriched for regulatory elements and that are flanked by digestion products (sticky ends) of restriction enzymes A and B to an isolation method of the invention, thereby isolating a collection of single-stranded DNA molecules comprising the regulatory elements, in a form suitable for sequencing at least a portion of each of the DNA molecules, and

sequencing at least a portion of at least one of the DNA molecules.

Preferably, the dsDNA molecules are about 100-400 bp in length.

In a sequencing method of the invention, the collection of dsDNA molecules may be obtained by a method comprising (a) digesting chromatin from the cell with restriction enzyme A, under conditions effective to cleave the accessible regions of the chromatin on the average of one time (preferably, no more than one time); (b) deproteinizing the digested chromatin; and (c) digesting the deproteinized DNA substantially to completion with restriction enzyme B, thereby generating a collection of dsDNA molecules that are enriched for regulatory elements and that are flanked by digestion products of restriction enzymes A and B. With regard to step (c), the digest with restriction enzyme B does not necessarily have to go to completion. A digest that goes “substantially” to completion is one that provides a sufficient amount of the doubly digested DNA to be usable for the method (e.g., for sequencing the DNA). For example, “substantially” to completion may be, e.g., about 90%-100% digestion. The term “about” as use herein refers to plus of minus 10%. Thus, “about” 90% encompasses 81%-99%. In order to substantially reduce non-specific cleavage due to random shearing, the method can further comprise embedding the DNA digested with restriction enzyme A in an agarose plug, and carrying out the deproteinization and digestion with restriction enzyme B in the agarose plug. Preferably, the dsDNA molecules are about 100-400 bp in length. Fragments of the desired size may be obtained by any of a variety of methods, including electrophoresis through an agarose gel.

In one embodiment of the invention, the DNA molecule is sequenced for about 30 bases (e.g., using the Solexa method), in another for about 100 bases or 230 bases (e.g., using the 454 Genome Sequencer 20 or FLX, respectively). Each of the DNA molecules in the collection may be sequenced from the sequencing primer site in adaptor A, or from the sequencing primer sites in both adaptor A and adaptor B.

In one embodiment of the invention,

the DNA molecules that are enriched for regulatory elements are about 100-400 bp in length; and adaptor B comprises, at its 5′ end, a biotin molecule, the method comprising

a) ligating adaptors A and B to the collection of dsDNA molecules, thereby forming ligated, partially dsDNA molecules,

b) immobilizing (attaching) the ligated, partially dsDNA molecules on magnetic streptavidin-coated beads, via the biotin molecules,

c) separating (removing) non-immobilized (unbound) DNA from the magnetic streptavidin-coated beads,

d) treating the ligated, partially dsDNA molecules which are immobilized on the beads under conditions effective to till in single-stranded regions, thereby generating fully dsDNA molecules,

e) melting the fully dsDNA molecules to release non-biotinylated, non-immobilized DNA strands from the beads, and

f) sequencing at least a portion of each of the released ssDNA molecules, using the sequencing primer in either adaptor A or in adaptor B (preferably using the sequencing primer sequence in adaptor A).

The method may further comprise

attaching the released single-stranded DNA molecules to sequencing beads under conditions such that no more that one single-stranded DNA molecule is attached to each bead,

placing each sequencing bead in a separate compartment (microreactor) and amplifying the DNA attached thereto by emulsion PCR (emPCR), and

sequencing the amplified DNA in a high throughput sequencing apparatus (e.g. a 454instrument), in a 5′-3′ direction, starting from the sequence priming region of adaptor A and/or of adaptor B.

In one embodiment of the invention, restriction enzyme A is a combination of HpaII, MseI and NlaIII. In this embodiment, at least about 94% of the accessible (e.g., regulatory, such as transcriptionally active) sequences of the cell can be sequenced.

In one embodiment of the invention, restriction enzyme A cuts in an accessible region of chromatin, so that the portion of the DNA of interest that is sequenced beginning with the sequencing primer region in adaptor A is from the accessible region of the DNA in chromatin.

Continuation that the isolated sequenced DNAs are from accessible regions can be accomplished, for example, by conducting DNAse hypersensitive site mapping in the vicinity of any accessible region sequence obtained by a method disclosed herein. Co-localization of a particular insert sequence with a DNAse hypersensitive site validates the identity of the insert as an accessible regulatory region.

A method of the invention can be utilized for a variety of purposes.

For example, a method of the invention can be used to define the chromatin architecture of a cell. In one embodiment, chromatin is treated by a method of the invention, and the sequences of the accessible regions of the chromatin are analyzed. This type of analysis can confirm the expected finding that spacers between nucleosomes are accessible to enzymatic digestion.

The regulatory regions can be mapped to identify which genes in a genome they regulate. The map locations of a large collection of such regions can be determined by comparing the sequences with genomic sequence databases.

The isolated accessible regions can be used to form collections or databases of accessible regions; generally the collections correspond to regions that are accessible for a particular cell. As used herein, the term “collection” refers to a pool of DNA fragments that have been isolated by a method of the invention.

The collections formed can represent accessible regions for a particular cell type or cellular condition. Thus, different collections can represent, for example, accessible regions for: cells that express a gene of interest at a high level, cells that express a gene of interest at a low level, cells that do not express a gene of interest, healthy cells, diseased cells, infected cells, uninfected cells, and/or cells at various stages of development. Alternatively or in addition, such individual collections can be combined to form a group of collections. Essentially any number of collections can be combined.

Typically, a group of collections contains at least 2, 5 or 10 collections, each collection corresponding to a different type of cell or a different cellular state. For example, a group of collections can comprise a collection from cells infected with one or more pathogenic agents and a collection from counterpart uninfected cells. Determination of the nucleotide sequences of the members of a group of collections can be used to generate a database of accessible sequences specific to a particular cell type.

In another embodiment, computer-based subtractive hybridization techniques can be used in the analysis of two or more collections of accessible sequences, obtained by any of the methods disclosed herein, to identify sequences that are unique to one or more of the collections. For example accessible sequences from normal cells can be subtracted from accessible sequences present in virus-infected cells to obtain a collection of accessible sequences unique to the virus-infected cells. Conversely, accessible sequences from virus-infected cells can be subtracted from accessible sequences present in uninfected cells to obtain a collection of sequences that become inaccessible in virus-infected cells. Such unique sequences obtained by subtraction can be used to generate databases. Methods of such difference analysis are conventional and well-known to those of skill in the art.

Sequences of accessible regions that are unique to a cell that expresses high levels of a gene of interest (“functional accessible sequences”) are important for the regulation of that gene. Similarly, sequences of accessible regions that are unique to a cell expressing little or none of a particular gene product are also functional accessible sequences and can be involved in the repression of that gene.

In addition, the presence of tissue-specific regulatory elements in a gene provide an indication of the particular cell and tissue type in which the gene is expressed. Genes sharing a particular accessible site in a particular cell, and/or sharing common regulatory sequences, are likely to undergo coordinate regulation in that cell.

Furthermore, association of regulatory sequences with EST expression profiles provides a network of gene expression data, linking expression of particular ESTs to particular cell types.

Thus, described herein are methods of monitoring how one or more conditions, disease states or candidate effector molecules (e.g., drugs) affect the nature of accessible regions, particularly regulatory accessible regions. The term “nature of accessible regions” is used to refer to any characteristic of an accessible region including, but not limited to, the location and/or extent of the accessible regions. To determine the effect of one or more drugs on these regions, accessible regions are compared between control (e.g., normal or untreated) cells and test cell (e.g., a diseased cell or a cell exposed to a candidate regulatory molecule such as a drug, a protein, etc.), using any of the methods described herein. Such comparisons can be accomplished with individual cells or using collections of accessible regions. The unique and/or modified accessible regions can also be sequenced to determine if they contain any potential known regulatory sequences. In addition, the gene related to the regulatory accessible region(s) in test cells can be readily identified using conventional methods.

Thus, candidate regulatory molecules can also be evaluated for their direct effects on chromatin, accessible regions and/or gene expression, as described herein. Such analyses will allow the development of diagnostic, prophylactic and therapeutic molecules and systems.

When evaluating the effect of a disease or condition, normal cells are compared to cells known to have the particular condition or disease. Disease states or conditions of interest include, but are not limited to, cardiovascular disease, cancers, inflammatory conditions, graft rejection and/or neurodegenerative conditions. Similarly, when evaluating the effect of a candidate regulatory molecule on accessible regions, the locations of accessible regions in any given cell can be evaluated before and after administration of a small molecule. As will be readily apparent from the teachings herein, concentration of the candidate small molecule and time of incubation can, of course, be varied. In these ways, the effect of the disease, condition, and/or small molecule on changes in chromatin structure (e.g., accessibility) or on transcription (e.g., through binding of RNA polymerase II) is monitored.

The methods are applicable to various cells, for example, human cells, animal cells, plant cells, fungal cells, bacterial cells, viruses and yeast cells. Another example of the application of these methods is in diagnosis and treatment or human and animal pathogens (e.g., bacteria, viral or fungal pathogens).

Collections of sequences corresponding to accessible regions can be utilized to conduct a variety of different comparisons to obtain information on the regulation of cellular transcription. Such collections of sequences can be obtained as described above and used to populate a database, which in turn is utilized in conjunction with conventional computerized systems and programs to conduct the comparison.

In certain methods for analysis of accessible regions and characterization of cells with respect to their accessible regions, a collection of accessible region sequences from one cell is compared to a collection of accessible region sequences from one or more other cells. For example, databases from two or more different cell types can be compared, and sequences that are unique to one or more cell types can be determined. These types of comparison can yield developmental stage-specific regulatory sequences, if the different cell types are from different developmental stages of the same organism. They can yield tissue-specific regulatory sequences, if the different cell types are from different tissues of the same organism. They can yield disease-specific regulatory sequences, if one or more of the cell types is from a diseased tissue and one of the cell types is the normal counterpart of the diseased tissue. Diseased tissue can include, for example, tissue that has been infected by a pathogen, tissue that has been exposed to a toxin, neoplastic tissue, and apoptotic tissue. Pathogens include bacteria, viruses, protozoa, fungi, mycoplasma, prions and other pathogenic agents as are known to those of skill in the art. Hence, comparisons can also be made between infected and uninfected cells to determine the effects of infection on host gene expression. In addition, accessible regions in the genome of an infecting organism can be identified, isolated and analyzed according to the methods disclosed herein. Those skilled in the art will recognize that a myriad of other comparisons can be performed.

Accessible sequences identified by a method of the invention can be mapped with regard to genes and coding regions. A collection of nucleotide sequences of accessible regions in a particular cell type is useful in conjunction with the genome sequence of an organism of interest. In one embodiment, information on regulatory sequences active in a particular cell type is provided. Although the sequences of regulatory elements are present in a genome sequence, they may not be identifiable (if homologos sequences are not known) and, even if they are identifiable, the genome sequence provides no information on the tissue(s) and developmental stage(s) in which a particular regulatory sequence is active in regulating gene expression. However, comparison of a collection of accessible region sequences from a particular cell with the genome sequence of the organism from which the cell is derived provides a collection of sequences within the genome of the organism that are active, in a regulatory fashion, in the cell type from which the accessible region sequences have been derived. This analysis also provides information on which genes are active in the particular cell, by allowing one to identify coding regions in the vicinity of accessible regions in that cell.

In addition, the aforementioned comparison can be utilized to map regulatory sequences onto the genome sequence of an organism. Since regulatory sequences are often in the vicinity of the genes whose expression they regulate, identification and mapping of regulatory sequences onto the genome sequence of an organism can result in the identification of new genes, especially those whose expression is at levels too low to be represented in EST databases. This can be accomplished, for example, by searching regions of the genome adjacent to a regulatory region (mapped as described above) for a coding sequence, using methods and algorithms that are well-known to those of skill in the art. The expression of many of the genes thus identified will be specific to the cell from which the accessible region database was derived. Thus, a further benefit is that new probes and markers, for the cells from which the collection of accessible regions was derived, are provided.

In addition to comparing the collection of polynucleotides against the entire genome, the sequences can also be compared against shorter known sequences such as intergenic regions, non-coding regions and various regulatory sequences, for example.

A method of the invention can also be used to characterize diseases. Comparisons of collections of accessible region sequences with other known sequences can be used in the analysis of disease states. For instance, collections such as databases of regulatory sequence are also useful in characterizing the molecular pathology of various diseases. As one example, if a particular single nucleotide polymorphism (SNP) is correlated with a particular disease or set of pathological symptoms, regulatory sequence collections or databases can be scanned to see if the SNP occurs in a regulatory sequence. If so, this result suggests that the regulatory sequence and/or the protein(s) which binds to it, are involved in the pathology of the disease. Identification of a protein that binds differentially to the SNP-containing sequence in diseased individuals compared to non-diseased individuals is further evidence for the role of the SNP-containing regulatory region in the disease. For example, a protein may bind more or less avidly to the SNP-containing sequence, compared to the normal sequence.

In other methods, comparisons can be conducted to determine correlation between microsatellite amplification and human disease such as for example, human hereditary neurological syndromes, which are often characterized by microsatellite expansion in regulatory regions of DNA. Other comparisons can be conducted to identify the loss of an accessible region, which can be diagnostic for a disease state. For instance, loss of an accessible region in a tumor cell, compared to its non-neoplastic counterpart, could indicate the lack of activation of a tumor suppressor gene in the tumor cell. Conversely, acquisition of an accessible region, as might accompany oncogene activation in a tumor cell, can also be an indicator of a disease state.

Comparisons can also be made to gene expression profiles. A collection of accessible sites that is specific to a particular cell can be compared with a gene expression profile of the same cell, such as is obtained by DNA microchip analysis. For example, serum stimulation of human fibroblasts induces expression of a group of genes (that are not expressed in untreated cells), as is detected by microchip analysis. Identification of accessible regions from the same serum-treated cell population can be accomplished by any of the methods disclosed herein. Comparison of accessible regions in treated cells with those in untreated cells, and determination of accessible sites that are unique to the treated cells, identifies DNA sequences involved in serum-stimulated gene activation.

Determining the location and/or sequence of accessible regions in a given cell can also be useful in pharmacogenomics (i.e. the identification of drug targets).

Pharmacogenomics (sometimes termed pharmacogenetics) refers to the application of genomic technology in drug development and drug therapy. In particular, pharmacogenomics focuses on the differences in drug response due to heredity and identifies polymorphisms (genetic variations) that lead to altered systemic drug concentrations and therapeutic responses. See, e.g., Eichelbaum, M. (1996) Clin. Exp. Pharmacol. Physiol. 23, 983 985 and Linder, M. W. (1997) Clin. Chem. 43, 254 266. The term “drug response” refers to any action or reaction of an individual to a drug, including, but not limited to, metabolism (e.g., rate of metabolism) and sensitivity (e.g., allergy, etc). Thus, in general, two types of pharmacogenetic conditions can be differentiated: genetic conditions transmitted as a single factor altering the way drugs act on the body (altered drug action) and genetic conditions transmitted as single factors altering the way the body acts on drugs (altered drug metabolism).

On a molecular level, drug metabolism and sensitivity is controlled in part by metabolizing enzymes and receptor proteins. In other words, a molecular change in a metabolic enzyme can cause a drug to be either slowly or rapidly metabolized. This can result in overabundant or inadequate amounts of drug at the receptor site, despite administration of a normal dose. Exemplary enzymes involved in drug metabolism include: cytochrome P450s; NAD(p)H quinone oxidoreductase; N-acetyltransferase and thiopurine methyltransferase (TPMT). Exemplary receptor proteins involved in drug metabolism and sensitivity include beta2-adrenergic receptor and the dopamine D3 receptor. Transporter proteins that are involved in drug metabolism include but are not limited to multiple drug resistance-1 gene (MDR-1) and multiple drug resistance proteins (MRPs).

Genetic polymorphism (e.g., loss of function, gene duplication, etc.) in these genes has been shown to have effects on drug metabolism. For example, mutations in the gene TPMT, which catalyzes the S-methylation of thiopurine drugs (i.e., mercaptopurine, azathioprine, thioguanine), can cause a reduction in its activity and corresponding ability to metabolize certain cancer drugs. Lack of enzymatic activity causes drug levels in the serum to reach toxic levels.

The methods of identifying accessible regions described herein can be used to evaluate and predict an individual's unique response to a drug by determining how the drug affects chromatin structure. In particular, alterations to accessible regions, particularly accessible regions associated with genes involved in drug metabolism (e.g., cytochrome P450, N-acetyltransferase, etc.), in response to administration of drugs can be evaluated in an individual subject: Accessible regions are identified, mapped and compared as described herein. For example, an individual's accessible region profile in one or more genes involved in drug metabolism can be obtained. Regulatory accessible region patterns and corresponding regulation of gene expression patterns of individual patients can then be compared in response to a particular drug to determine the appropriate drug and dose to administer to the individual.

Thus, identification of alterations in accessible regions in a subject will allow for targeting of the molecular mechanisms of disease and, in addition, design of drug treatment and dosing strategies that take variability in metabolism rates into account. Optimal dosing can be determined at the initiation of treatment, and potential interactions, complications, and response to therapy can be anticipated. Clinical outcomes can be improved, risk for adverse drug reactions (ADRs) will be minimized, and the overall costs for managing these reactions will be reduced. Pharmacogenomic testing can optimize the drug dose regimen for patients before treatment or early in therapy by identifying the most patient-specific therapy that can reduce adverse events, improve outcome, and decrease health costs.

In addition, sequence analysis and identification of regulatory binding sites in accessible regions can also be used to identify drug targets; potential drugs; and/or to modulate expression of a target gene. Such methods can be used in any suitable cell, including, but not limited to, human cells, animal cells (e.g., farm animals, pets, research animals), plant cells, and/or microbial cells. In plants, drug targets and effector molecules can be identified for their effects on herbicide resistance, pathogens, growth, yield, compositions (e.g., oils), production of chemical and/or biochemicals (e.g., proteins including vaccines). Methods of identifying drug targets can also find use in identifying drugs which may mediate expression in animal (including human) cells. In certain animals, for instance cows or pigs, drug targets are identified by determining potential regulatory accessible regions in animals with the desirable traits or conditions (e.g., resistance to disease, large size, suitability for production of organs for transplantation, etc.) and the genes associated with these accessible regions. In human cells, drug targets for many disease processes can be identified.

A method of the invention for isolating ssDNA molecules in a form suitable for sequencing can also be applied to other uses. For example, one or more of the single-stranded DNA molecules from regulatory regions can be amplified, rendered double-stranded, and characterized, e.g. to determine what protein components of a cell, such as transcription factors, bind to the regulatory region. In one application, the dsDNAs are attached to a matrix for affinity chromatography; a nuclear protein extract from a cell is passed through the column; the column is extensively washed; and proteins that have been bound to the column are eluted. The eluted proteins can then be characterized by conventional methods, such as Western blotting, 2-D electrophoresis, mass spectrometry analysis, etc. In another application, the collection of dsDNAs is passed through an affinity column containing proteins of interest, such as transcription factors. DNAs which bind specifically to the protein can then be eluted and characterized, e.g. sequenced.

A method of the invention can be used to prepare nucleic acid that can be used, without further purification, for any purpose and in any manner that nucleic acid cloned or amplified by known methods can be used. For example, the nucleic acid can be probed, cloned, transcribed, amplified, stored, or be subjected to hybridization, denaturation, restriction, haplotyping or microsatellite analysis or to a variety of SNP typing techniques.

One aspect of the invention is a DNA molecule (e.g., an intermediate in an isolation method of the invention), which is a partially dsDNA molecule that comprises, starting from the 5′ end,

a) a biotin molecule,

b) a single-stranded portion comprising a PCR priming region and a sequence priming region,

c) a double-stranded portion with a composite sequence composed of the digestion product of restriction enzyme A and a compatible sequence,

d) a dsDNA molecule of interest (e.g., from a transcriptionally active, regulatory region of chromatin),

e) a double-stranded portion with a composite sequence composed of the digestion product of restriction enzyme B and a compatible sequence, and

f) a single-stranded portion comprising a sequence priming region and a PCR priming region.

Another aspect of the invention is a ssDNA molecule which comprises, starting from the 5′ end,

a) a PCR priming region,

b) a sequence priming region,

c) a sequence that is compatible with the digestion product of restriction enzyme B,

d) a DNA molecule of interest (e.g., from a transcriptionally active, regulatory region of chromatin),

e) a sequence that is the digestion product of restriction enzyme A,

f) a sequence priming region, and

g) a PCR priming region.

Any combination of the materials useful in the disclosed methods can be packaged together as a kit for performing any of the disclosed methods.

In one embodiment, the kit comprises

a) a first partially duplex adaptor, adaptor A, which comprises, in the 5′ to 3′ direction, and in the following order, a single-stranded portion comprising a PCR priming region, a sequence priming region, and a double-stranded portion with a single-stranded overhang that is compatible with the digestion product of restriction enzyme site A, and

b) a second partially duplex adaptor, adaptor B, which comprises, starting at the 5′ end, an attachment agent (e.g. biotin), a single-stranded portion comprising a PCR priming region, a sequence priming region, and a double-stranded portion with a single-stranded overhang that is compatible with the digestion product of restriction enzyme site B.

In variations of a kit of the invention, restriction enzyme A comprises HpaII, MseI and/or NlaIII, and restriction enzyme B is an enzyme that recognizes a 4 bp recognition sequence; or restriction enzyme A comprises HpaII, MseI and NlaIII, and restriction enzyme B is an enzyme that recognizes a 4 bp recognition sequence (e.g. Sau3A I). In a preferred embodiment, a kit of the invention comprises, as restriction enzyme A, HpaII, MseI and NlaIII, and as the 4 bp recognition sequence, Sau3A I.

Enzymes necessary for the disclosed methods can also be components of such kits. A skilled worker will recognize components of kits suitable for carrying out any of the methods of the invention. Optionally, the kits comprise instructions for performing the method. Kits of the invention may further comprise suitable buffers, or the like, containers, or packaging materials. The reagents of the kit may be in containers in which the reagents are stable, e.g., in lyophilized form or stabilized liquids. The reagents may also be in single use form, e.g., in a form for the isolation of accessible regions from the chromatin of a cell.

In the foregoing and in the following examples, all temperatures are set forth in uncorrected degrees Celsius; and, unless otherwise indicated, all parts and percentages are by weight.

### EXAMPLES

### Example I

### Introduction

We have developed a rapid tag based approach for identifying regulatory DNA elements in human cells genome-wide using restriction enzymes. This methodology necessitates a large number of sequence reads for an accurate quantitative measure of functional sequence. High throughput sequence technology, such as the 454 sequencing technology, affords a large number of sequence reads which enable the rapid and comprehensive determination of the regulatory DNA in any particular cell type.

In these Examples, we show the preparation of functional DNA from CD34 and differentiated cells using restriction digests with NlaIII in chromatin preparations followed by Sau3A digests and size fractionation to identify fragments between 100-400 bp for sequencing. These DNA fragments are then ligated to modified (biotin) DNA adaptors and purified on streptavidin coated beads for subsequent processing through the standard 454 sequencing methodology. We localized greater than 60% of the 200,000-300,000 reads generated from each run on the genome sequence, the non-localized reads being >95% repeat sequence. Some 20-40% of the localized reads were found in overlapping clusters of two or more reads indicating a large number of genomic regions (>12,000) may be involved in gene regulation. We established that greater than 80% of these regions are DNase 1 hypersensitive (n=40).

This method provides a comprehensive, unbiased, high throughput approach for the detection of regulatory DNA in a cell via direct sequencing

A common feature of the regions of the genome that regulated the transcription of genes is their steric accessibility to enzymatic degradation. The preparation of such regulatory regions can be accomplished with restriction enzymes, making it possible to identify promoters and enhancer sequence regions from the chromatin architecture in a nucleus. We provide a global view of these regions by cutting and sequencing these domains in a high throughput manner using the GS20 454 analyzer. It should be noted that in this Example, the inventors used the GS20 instrument, which generates 100 base reads on average. An improved version of the 454 apparatus, the GS FLX instrument, allows for considerably longer reads.

### Example II

### Materials and Methods

A. Sample preparation

Chromatin preparation of CD34+ and myeloid cells

Cut Accessible DNA (1^{st} restriction enzyme action)

Prevent Degradation (agarose plug)

Controlled Shearing (2^{nd} restriction enzyme action).

### B. Purification and Sequencing

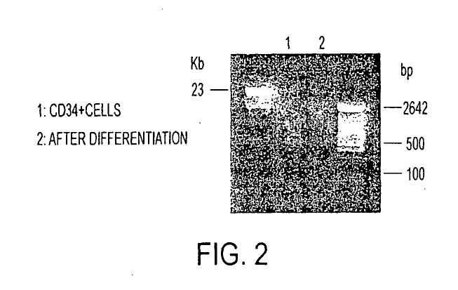

The sample was subjected to agarose gel purification to generate fragments in the size range 100-400 bp, as shown in FIG. 2.

Double restricted fragments were purified (isolated) using modified 454 PCR+sequencing adaptors with biotin tag (as described herein) on streptavidin coated magnetic beads, as illustrated in FIG. 1.

### C. Blast Mapping of Sequence Fragments

Fragments containing repeat sequence identified by RepeatMasker for more than 50% of their length were removed and the remaining fragments were aligned by BLAST to the human genome (NCB1 35). All unique or best hits alignments were identified and overlapping regions were collapsed to identify non redundant genomic spans. The 5′ most location of fragments are noted for all reliably mapped cases that contain a bona fide NlaIII recognition sequence at the 5′ end. This represents the number of NlaIII-hypersensitive sites from a particular DNA sample.

### III. Results

### A. Sensitivity and Localization of Fragments in the Genome

Greater than 99.6% of amplified and sequenced fragments contain an NlaIII recognition sequence at the 5′ end indicating that the process is highly selective for the authentic NlaIII cut site. A summary of the run statistics and mapping results in shown in Table 1.

TABLE 1

| | | | Diff - 1st | Diff - 2nd |
|---|---|---|---|---|
| | Naked | CD34 | run | run |
| Total number fragments | 323,630 | 217,378 | 259,298 | 283,703 |
| fragment aligning | 179,835 | 121,966 | 138,175 | 150,478 |
| uniquely | | | | |
| fragments single best hit | 31,823 | 19,251 | 23,804 | 25,860 |
| Total aligning | 211,658 | 141,217 | 161,979 | 176,338 |
| % aligning | 65.4 | 65.0 | 62.5 | 62.2 |
| Not aligning | 111,972 | 76,161 | 97,319 | 107,365 |
| % not aligning | 34.6 | 35.0 | 37.5 | 37.8 |
| repeat containing | 107,191 | 72,850 | 94,097 | 102,708 |
| sequences in those | | | | |
| not aligning | | | | |
| % not aligning that | 95.7 | 95.7 | 96.7 | 95.7 |
| are repeal | | | | |

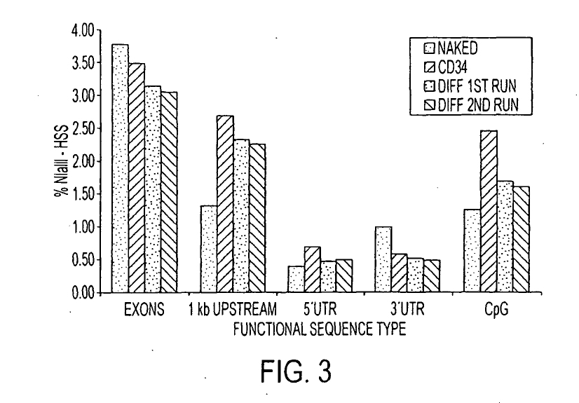

We found that CD34 and myeloid cells have an over-representation of NLA-hypersensitive sites in the region 1 kb upstream of gene transcription start sites, 5′ UTR and CpG domains. These sites are under-represented in exons and 3′ UTR. (Ensembl annotation version 31). These findings are shown in FIG. 3.

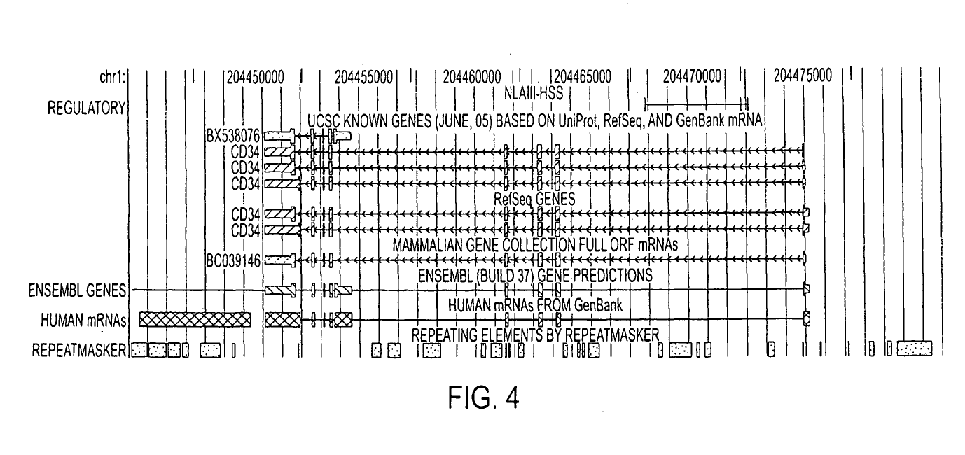

An example of the CD34 gene showing three hypersensitive sites in the first intron identified from CD34+ cells is shown in FIG. 4. These sites were not found in both runs from myeloid cells. 20-40% of the NlaIII hypersensitive sites are in neighboring clusters (<100 bp apart) containing 2 sites or more, highlighting the prospect that between 13,000-25,000 genomic regions are accessible per cell type.

### B. Fragments are Adjacent to Transcription Start Sites and 5′ UTR Regions

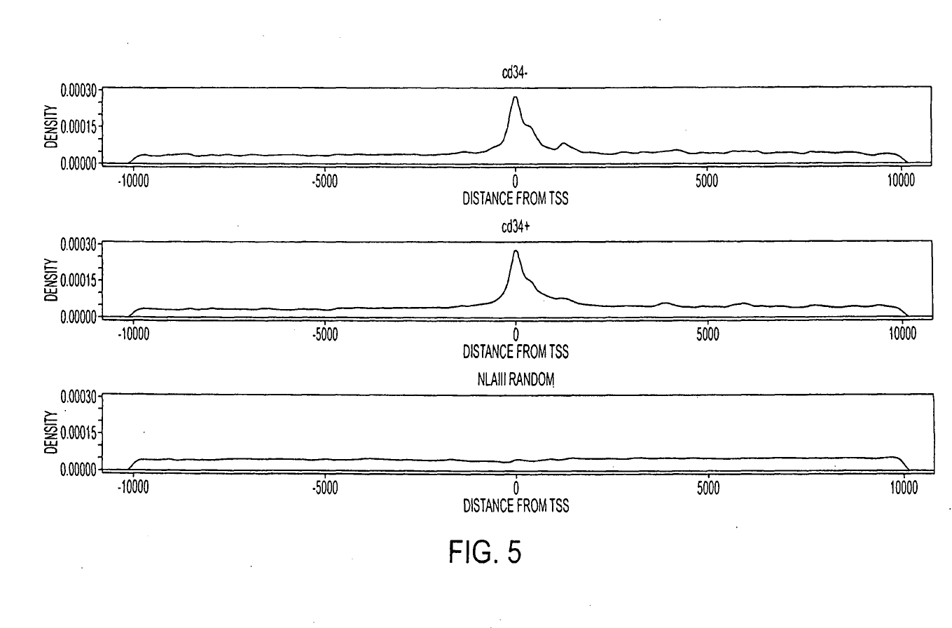

Evidence that fragments are adjacent to transcription start sites and 5′ UTR regions is shown in FIG. 5.

### C. Non-Mapped Fragments are Primarily L1-LINE, LTR and SINEs

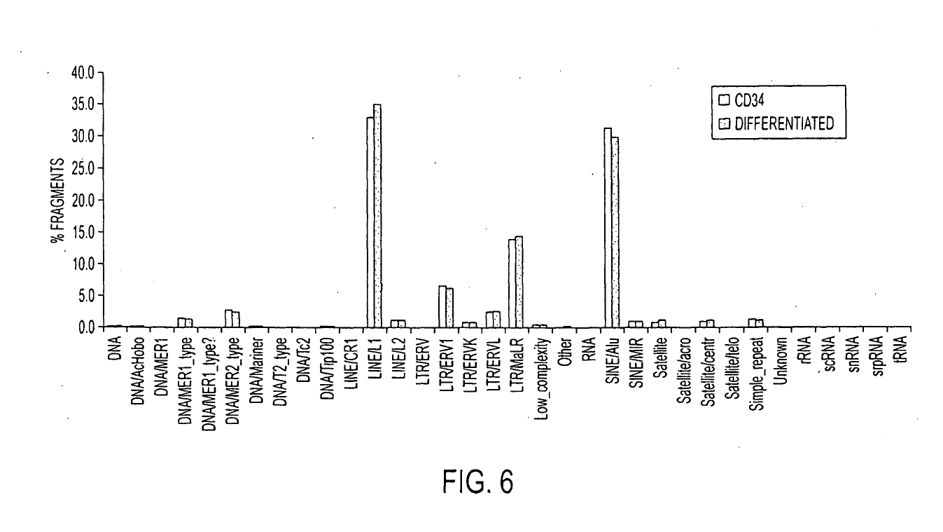

Evidence that the non-mapped fragments are primarily L1-LINE, LTR and SINEs is presented in FIG. 6.

### D. Clone Validation Using Hypersensitivity Assays

Using quantitative PCR, we showed that 80% of regions identified as containing NlaIII accessible site are also DNaseI hypersensitive. Forty target regions were tested in an unbiased manner contain either single or multiple NlaIII accessible sites.

### E. Conclusions

The chromatin extraction methodology employs a non biased (non-antibody based) means of identifying exposed DNA segments accessible within the context of chromatin.

Up to 250,000 genomic regions can be identified in one 454 run.

These regions are typically found in 1 kb upstream, 5′ UTR, CpG domains and are under-represented in exons and 3′ UTR.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. Provisional Application No. 60/851,292, filed Oct. 13, 2006, and in the figures are hereby incorporated in their entirety by reference.

1. A method for isolating a DNA molecule of interest in a form suitable for sequencing at least a portion of the DNA by a high throughput sequencing method, comprising
digesting double-stranded (ds)DNA with two different restriction enzymes, A and B, that produce sticky ended cleavage products, to generate a ds form of the DNA molecule of interest that is bounded by the two restriction enzyme cleavage products, and
attaching to each end of the DNA molecule of interest an adaptor molecule which comprises at one end a sticky end that is compatible with either the restriction enzyme A cleavage product or the restriction enzyme B cleavage product, and which also comprises one or more sequences and/or elements, including a sequence priming region, that allow the DNA of interest to be sequenced with a high throughput sequencing apparatus.

2. The method of claim 1, further comprising
converting the ds form of the DNA molecule of interest which is flanked by the adaptors to single-stranded (ss)DNA;
amplifying the ssDNA; and
sequencing the amplified DNA with a high throughput sequencing apparatus.

3. The method of claim 1, wherein the high throughput sequencing apparatus is a 454 instrument and the sequencing method is a modification of conventional 454 technology, wherein instead of the conventional adaptor used for 454 technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product.

4. The method of claim 3, further wherein, after the adaptors have been added to the ds form of the DNA of interest,
the ds form of the DNA of interest is bound to a surface via an attachment agent that is present at the end of one of the adaptors;
the bound, ds form of the DNA of interest is melted and single-stranded molecules of the DNA of interest are released from the surface and collected;
the released ssDNA is bound to a capture bead, via a sequence that is present in one of the adaptors, under conditions such that no more than one ssDNA molecule is attached to each bead;
the ssDNA bound to the capture bead is amplified by PCR, via a PCR priming site that is present in one of the adaptors; and
at least a portion of the amplified DNA is sequenced, via a sequence priming region that is part of one of the adaptors, using 454 technology.

5. The method of claim 1, wherein the high throughput sequencing method is a modification of conventional Solexa technology, wherein instead of the conventional adaptor used for Solexa technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product.

6. The method of claim 5, further wherein, after the adaptors have been added to the ds form of the DNA of interest,
the ds form of the DNA of interest is amplified by PCR to increase its copy number;
the amplified DNA is denatured to form single strands, the single strands are diluted, and single copies of the single-stranded DNA are bound, via a sequence that is present in one of the adaptors, to one of a plurality of oligonucleotides located at definable positions on a surface, under conditions such that no more than one DNA molecule is bound at each position on the surface;
the bound ssDNA is amplified by bridge amplification, using sequences that are present in the adaptors, to form a clonal cluster on the surface; and
at least a portion of the bound, amplified DNA in the clusters is sequenced, via a sequence priming region that is part of one of the adaptors, using Solexa technology.

7. The method of claim 1, wherein the high throughput sequencing apparatus is an ABI instrument and the sequencing method is a modification of the conventional SOLiD™ method, wherein instead of the conventional adaptor used for the SOLiD™ technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product.

8. The method of claim 7, further wherein, after the adaptors have been added to the ds form of the DNA of interest,
the ds form of the DNA of interest is circularized by ligating each end of the dsDNA of interest to a DNA segment, wherein a sequence at the free end of each of the adaptors is compatible with a sequence at one of the ends of the DNA segment;
the circularized DNA is contacted with the restriction enzyme EcoP151, under conditions such that the restriction enzyme binds to a recognition sequence that is present in each adaptor, and cuts downstream at a distance within the DNA of interest, to generate a linear double-stranded molecule that comprises, starting at one end of the molecule, about 25 bp from one end of the DNA of interest, a first adaptor, the DNA segment, a second adaptor, and about 25 bp from the other end of the DNA of interest;
the double-stranded linear molecule is ligated, at each end, to a molecule which comprises a PCR priming site, and the resulting dsDNA is amplified by PCR to increase its copy number;
the amplified DNA is denatured to form single strands, the single strands are diluted, and single copies of the single-stranded DNA are bound, via a sequence that is present in one of the adaptors, to a capture bead;
the bound ssDNA is amplified by PCR, via a PCR priming site that is present in one of the adaptors; and
at least a portion of the amplified DNA is sequenced, via a sequence priming region that is part of one of the adaptors, using ABI SOLiD™ technology.

9. The method of claim 1, wherein the DNA of interest is from an accessible region of chromatin.

10. The method of claim 9, wherein the accessible region of chromatin comprises regulatory and/or transcriptionally active sequences.

11. The method of claim 3, further comprising
a) contacting the ds form of the DNA of interest with two adaptors:
i) a first partially duplex adaptor, adaptor A, which comprises, in the 5′ to 3′ direction, in the following order, a single-stranded portion comprising a PCR priming region and a sequence priming region, and then a double-stranded portion with a single-stranded overhang that is compatible with the digestion product of restriction enzyme A, and
ii) a second partially duplex adaptor, adaptor B, which comprises, starting at the 5′ end, an attachment agent, a single-stranded portion comprising a PCR priming region, a single-stranded sequence priming region, and a double-stranded portion with a single-stranded overhang that is compatible with the digestion product of restriction enzyme B,
under conditions that are effective to join the ds form of the DNA of interest to the two adaptors, to ligate nicks thus formed, and to attach the joined, ligated, partially dsDNA molecule to a surface;
b) removing the joined, partially dsDNA molecule attached to the surface from unbound DNA molecules;
c) subjecting the joined, partially dsDNA molecule attached to the surface to conditions effective for filling in single-stranded regions, thereby forming a full-length ds DNA attached to the surface; and
d) separating the strands of the DNA molecule bound to the surface to release from the surface the single-full-length strand of the DNA which lacks the attachment agent, thereby isolating a single-stranded DNA molecule comprising the sequence of the DNA of interest, in a form suitable for sequencing at least a portion of the DNA of interest.

12. The method of claim 11, wherein the surface is a bead, the attachment agent is biotin, the surface of the bead comprises streptavidin, and the binding is achieved by interaction of the biotin and the streptavidin.

13-20. (canceled)

21. The method of claim 1, wherein restriction enzyme A digests accessible regions in chromatin and is a combination (cocktail) comprising
a) a methylation-sensitive enzyme whose recognition site contains a CG dinucleotide;
b) an enzyme that cuts sequences having solely A or T residues; and/or
c) an enzyme whose recognition site consists of a palindromic combination of A, G, C and T.

22-26. (canceled)

27. The method of claim 21, wherein restriction enzyme A is a combination consisting of HpaII, MseI, and NlaIII.

28. The method of claim 1, wherein restriction enzyme B has a recognition sequence of 4 bp.

29. The method of claim 28, wherein restriction enzyme B is Sau3A I and/or NlaIII.

30-33. (canceled)

34. A method for sequencing regulatory elements within a cell, comprising
digesting chromatin from the cell's nucleus with restriction enzyme A, under conditions effective to cleave the accessible regions of the chromatin on the average of one time,
deproteinizing the digested chromatin,
digesting the deproteinized DNA substantially to completion with restriction enzyme B, thereby generating a collection of double-stranded (ds)DNA molecules that are enriched for regulatory elements and that are flanked by digestion products of restriction enzymes A and B,
attaching to each end of the dsDNA molecules that are flanked by digestion products of restriction enzymes A and B an adaptor molecule which comprises at one end a sticky end that is compatible with either the restriction enzyme A cleavage product or the restriction enzyme B cleavage product, and which also comprises one or more sequences and/or elements, including a sequence priming region, that allow the DNA of interest to be sequenced with a high throughput sequencing apparatus,
converting the dsDNA molecules which are flanked by the adaptors to single-stranded (ss)DNA, thereby isolating a collection of single-stranded DNA molecules comprising the regulatory elements, in a form suitable for sequencing at least a portion of each of the DNA molecules;
amplifying the ssDNA; and
sequencing at least a portion of at least one of the amplified DNA molecules with a high throughput sequencing apparatus.

35-45. (canceled)

46. A partially dsDNA molecule which comprises, starting from the 5′ end,
a) a biotin molecule,
b) a single-stranded portion comprising a PCR priming region and a sequence priming region,
c) a double-stranded portion with a composite sequence composed of the digestion product of a restriction enzyme A and a compatible sequence,
d) a dsDNA molecule of interest,
e) a double-stranded portion with a composite sequence composed of the digestion product of a restriction enzyme B and a compatible sequence, and
f) a single-stranded portion comprising a sequence priming region and a PCR priming region, or
a ssDNA molecule which comprises, starting from the 5′ end,
a) a PCR priming region,
b) a sequence priming region,
c) a sequence that is compatible with the digestion product of restriction enzyme B,
d) a DNA molecule of interest,
e) a sequence that is the digestion product of restriction enzyme A,
f) a sequence priming region, and
g) a PCR priming region.

47. (canceled)

48. A kit that comprises
a) a first partially duplex adaptor, adaptor A, which comprises, in the 5′ to 3′ direction, and in the following order, a single-stranded portion comprising a PCR priming region, a sequence priming region, and a double-stranded portion with a single-stranded overhang that is compatible with the digestion product of restriction enzyme site A, and
b) a second partially duplex adaptor, adaptor B, which comprises, starting at the 5′ end, an attachment agent, a single-stranded portion comprising a PCR priming region, a sequence priming region, and a double-stranded portion with a single-stranded overhang that is compatible with the digestion product of restriction enzyme site B.

49-55. (canceled)

56. The method of claim 34, wherein,
a) the DNA is sequenced by a modification of conventional 454 technology, wherein instead of the conventional adaptor used for 454 technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product;
b) the DNA is sequenced by a modification of conventional Illumina-Solexa technology, wherein instead of the conventional adaptor used for Illumina-Solexa technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product; or
c) the high throughput sequencing apparatus is an ABI instrument and the DNA is sequenced by a modification of the conventional SOLiD™ method, wherein instead of the conventional adaptor used for the SOLiD™ technology, which binds to the DNA of interest via a blunt end, two adaptors are used, in one of which the blunt end of the conventional adaptor is replaced with a sequence that is compatible with the restriction enzyme A cleavage product, and in the other of which the blunt end of the conventional adaptor is replaced with a sequence that is compatible with the restriction enzyme B cleavage product.
